(19) United States
(12) Patent Application Publication
(10) Pub. No.: US 2024/0050332 A1
(43) Pub. Date: Feb. 15, 2024

(54) Oral Care Compositions and Methods

(71) Applicant: Colgate-Palmolive Company, New York, NY (US)

(72) Inventors: Carl MYERS, Wayne, NJ (US); Guofeng XU, Plainsboro, NJ (US); Shaoyi ZHANG, South Bound Brook, NJ (US); Saide TANG, Princeton, NJ (US)

(73) Assignee: Colgate-Palmolive Company, New York, NY (US)

(21) Appl. No.: 18/258,248

(22) PCT Filed: Dec. 20, 2021
(86) PCT No.: PCT/US2021/064401

§ 371 Date: Jun. 19, 2023

Related U.S. Application Data
(60) Provisional application No. 63/128,671, filed on Dec. 21, 2020.

(51) Int. Cl. A61K 8/21; A61Q 11/00; A61K 8/24; A61K 8/55

(52) U.S. Cl. CPC A61K 8/21; A61Q 11/00; A61K 8/24; A61K 8/55

(57) ABSTRACT

This application relates to novel aqueous oral care compositions useful for combining and delivering incompatible stannous fluoride, and/or stannous chloride, and/or stannous pyrophosphate and potassium salts, with a polyphosphate source, wherein the polyphosphate source comprises three or more phosphate moieties or units, in a high-water composition, for example, to provide effective caries prevention, protection against dental erosion, relief from dental hypersensitivity, and to treat or reduce malodor.

### FIELD OF THE INVENTION

This application relates to novel aqueous oral care compositions useful for combining and delivering incompatible stannous fluoride, stannous chloride, or stannous pyrophosphate and nitric acid or a water-soluble nitrate salts, in combination with one or more polyphosphate sources (e.g., wherein at least one of the polyphosphate sources comprises three or more phosphate moieties), in a high-water composition, for example, to provide effective caries prevention, protection against dental erosion, and relief from dental hypersensitivity and malodor.

### BACKGROUND

Dental plaque is a sticky biofilm or mass of bacteria that is commonly found between the teeth, along the gum line, and below the gum line margins. Dental plaque can give rise to dental caries and periodontal problems such as gingivitis and periodontitis. Dental caries tooth decay or tooth demineralization caused by acid produced from the bacterial degradation of fermentable sugar.

Oral care compositions which contain stannous ion sources exhibit excellent clinical benefits, particularly in the reduction of gingivitis. Certain stannous ion sources are known for use in clinical dentistry with a history of therapeutic benefits over forty years. However, until recently, its popularity has been limited by its instability in aqueous solutions. The instability of stannous salts in water is primarily due to the reactivity of the stannous ion (Sn²⁺). Stannous salts readily hydrolyze at a pH above 4, resulting in precipitation from solution. It has traditionally been thought that this formation of insoluble stannous salts results in a loss of therapeutic properties.

One common way to overcome the stability problems associated with stannous ions is to limit the amount of water in the composition to very low levels, or to use a dual phase system. Both of these solutions to the stannous ion problem have drawbacks. Low water oral care compositions can be difficult to formulate with desired rheological properties, and dual-phase compositions are considerably more expensive to manufacture and package. Thus, it is preferable to formulate a high-water composition which uses an alternative means to maintain stable efficacious stannous ion concentrations.

While it has been generally suggested that oral care compositions comprising stannous salts, fluoride salts, and polyphosphate could be prepared, many references do not take issue with or seem to be aware of the unique formulation difficulties which may be encountered, or avoid the issues by resorting to dual-component manufactures.

There is thus a need for novel oral compositions and methods that provide stable formulations of stannous and potassium salts, and, in certain aspects, are able to be used to provide effective caries prevention, protection against dental erosion, and relief from dental hypersensitivity and possibly malodor.

### BRIEF SUMMARY

Without being bound by theory, it is believed that a combination of stannous pyrophosphate, nitric acid or a soluble nitrate salt, and one or more polyphosphate sources, wherein the polyphosphate source comprises three or more phosphate moieties or units (e.g., 3 to 50 phosphate moieties or units), in high-water oral care composition results in stability of stannous, fluoride and nitrate in solution. In one aspect, the nitrate salt is an alkali metal nitrate (e.g., potassium nitrate) and the polyphosphate sources, wherein the polyphosphate source comprises three or more phosphate moieties is a hexametaphosphate (e.g., sodium hexametaphosphate), trimetaphosphate (e.g., sodium trimetaphosphate) or phytic acid. In some embodiments, the composition comprises at least 10% water, e.g., at least 50% or at least 75% w/w of water. In some embodiments, the composition has a pH above 6.0, e.g., of about 7. For example, the composition is a toothpaste (e.g., toothpaste gel) or a mouthwash.

Again, without being bound by theory, the compositions described herein allow a novel approach to stabilize stannous ions (e.g, from stannous fluoride, stannous chloride, or stannous pyrophosphate) in high water dentifrice systems, which allows the stannous ions to be both soluble and stable. Without being bound by theory, this improved solubility and stability of stannous ions allows them to be more readily available to, for example, provide effective caries prevention, protection against dental erosion, and relief from dental hypersensitivity and malodor.

The disclosure further provides methods of stabilizing stannous ion in an aqueous oral care composition formulating the composition with a nitrate ion source (e.g., potassium nitrate) and a polyphosphate source, wherein the polyphosphate source comprises three or more phosphate moieties (e.g., sodium hexametaphosphate, e.g., sodium trimetaphosphate, e.g., phytic acid, e.g, tetrasodium pyrophosphate) in a high-water composition (e.g., at least 10% w/w of water), optionally wherein the solution has a pH above 6.0 (e.g., about 7). U.S. application Ser. No. 16/840,857, incorporated by reference herein in its entirety, discloses the surprising discovery that a combination of stannous fluoride or stannous chloride, nitric acid or a soluble nitrate salt, and an alkali metal polyphosphate salt (e.g., sodium tetrapyrophosphate (TSPP)) in high-water oral care composition results in stability of stannous, fluoride and nitrate in solution.

It is also believed that the stabilization of stannous using nitrate and polyphosphate source according to the present disclosure can result in extremely clear and translucent toothpaste and gel compositions, which is a significant advance in toothpaste aesthetics.

The disclosure further provides single-component oral care composition packages comprising the compositions disclosed herein.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

As is usual in the art, the compositions described herein are sometimes described in terms of their ingredients, notwithstanding that the ingredients may disassociate, associate or react in the formulation. Ions, for example, are commonly provided to a formulation in the form of a salt, which may dissolve and disassociate in aqueous solution. It is understood that the invention encompasses both the mixture of described ingredients and the product thus obtained.

In a first aspect, the present disclosure provides a single-component oral care composition (Composition 1.0) comprising:
(i) a stannous ion source selected from: stannous fluoride, stannous chloride, stannous pyrophosphate and combinations thereof;
(ii) nitric acid or a water-soluble nitrate salt (e.g., potassium nitrate);
(iii) a polyphosphate source, wherein the polyphosphate source comprises three or more phosphate moieties or units ((e.g., from 3 to 50 units) (e.g., from 3 to 18 units) ((e.g., from 4 to 50 units) (e.g., from 4 to 18 units) (e.g., from 6 to 50 units) (e.g., from 6 to 18 units)) (e.g., sodium hexametaphosphate, sodium trimetaphosphate, sodium tetrametaphosphate or phytic acid); and
(iv) more than 10% water, by weight of the composition.

For example, the disclosure provides embodiments of Composition 1 as follows:
1.1 Composition 1.0, wherein the water-soluble nitrate salt is selected from an alkali or alkaline earth metal nitrate, or zinc nitrate, silver nitrate, or ammonium nitrate.
1.2 Composition 1.1, wherein the water-soluble nitrate salt is an alkali metal nitrate salt or an alkaline earth metal nitrate salt.
1.3 Composition 1.2, wherein the nitrate salt is selected from lithium nitrate, sodium nitrate, potassium nitrate, magnesium nitrate, and calcium nitrate.
1.4 Composition 1.3, wherein the nitrate salt is potassium nitrate.
1.5 Any foregoing composition, wherein the polyphosphate source comprising three or more phosphate moieties or units ((e.g., from 3 to 50 units) (e.g., from 3 to 18 units) ((e.g., from 4 to 50 units) (e.g., from 4 to 18 units) (e.g., from 6 to 50 units) (e.g., from 6 to 18 units)) is in a linear or ring arrangement (e.g., cyclic polyphosphates), or pendants of a polymer.
1.6 Any foregoing composition, wherein the polyphosphate source comprising three or more phosphate moieties is a polyphosphate source where the phosphates are pendent groups of a ring (e.g., phytic acid).
1.7 Any foregoing composition, wherein the polyphosphate source comprising three or more phosphate moieties is a phosphate source wherein the phosphates are pendent groups of a polymer (e.g., ethylene glycol methacrylate phosphate, vinylphosphonic acid, bis2-(methacryloyloxy)ethyl phosphate, 11-phosphonoundecyl acrylate, 2-methacryloyloxyethylphosphorcholine).
1.8 Any foregoing composition, wherein the wherein the polyphosphate source comprising three or more phosphate moieties or units is selected from: sodium trimetaphosphate, sodium tetrametaphosphate, sodium hexametaphosphate, phytic acid and combinations thereof.
1.9 Any foregoing composition, wherein the polyphosphate source comprising three or more phosphate moieties or units is selected from sodium hexametaphosphate, sodium trimetaphosphate, phytic acid and combinations thereof.
1.10 Any foregoing composition, wherein the composition further comprises a water-soluble alkali metal polyphosphate selected from a pyrophosphate, tripolyphosphate, tetraphosphate or hexametaphosphate.
1.11 The composition of 1.10, wherein the water-soluble alkali metal polyphosphate is a sodium or potassium polyphosphate.
1.12 The composition of 1.10 or 1.11, wherein the water-soluble alkali metal polyphosphate is selected from sodium pyrophosphate, potassium pyrophosphate, sodium tripolyphosphate and potassium tripolyphosphate.
1.13 Composition 1.12, wherein the sodium pyrophosphate salt is selected from sodium acid pyrophosphate (i.e., disodium pyrophosphate) and tetrasodium pyrophosphate.
1.14 The composition of 1.10-1.13, wherein the water-soluble nitrate salt is potassium nitrate and the water-soluble alkali metal polyphosphate salt is tetrasodium pyrophosphate.
1.15 Any foregoing composition, wherein the composition comprises stannous fluoride (e.g., stannous fluoride is the only source of stannous).
1.16 Any foregoing composition, wherein the composition comprises stannous chloride (e.g., stannous chloride is the only source of stannous).
1.17 Any foregoing composition, wherein the composition comprises stannous pyrophosphate (e.g., stannous pyrophosphate is the only source of stannous).
1.18 Any foregoing composition, wherein the composition comprises one or more of stannous fluoride, stannous chloride, and stannous pyrophosphate (e.g., one or more in combination).
1.19 Any foregoing compositions, wherein the composition further comprises an additional stannous ion source.
1.20 Any foregoing composition, wherein the composition comprises a molar ratio of polyphosphate source comprising three or more phosphate moities (e.g., sodium hexametaphosphate, sodium trimetaphosphate, sodium tetrametaphosphate or phytic acid) to stannous chloride, stannous fluoride, or stannous pyrophosphate of at least 1:1, e.g., 1:1 to 5:1, or 1:1 to 4:1, or 1:1 to 3:1, or 1:1 to 2:1, or 1.5:1 to 5:1, or 2:1 to 5:1, or 2:1 to 4:1, or 2:1 to 3:1, or about 1:1.
1.21 Any foregoing composition, wherein the composition comprises a molar ratio of nitric acid or water-soluble nitrate salt (e.g., potassium nitrate) to stannous chloride, stannous fluoride, or stannous pyrophosphate of at least 0.01:1, e.g., 0.01:1 to 20:1, 0.01:1 to 0.1:1, or 0.03:1, or 0.3:1 or 0.5:1 to 20:1, or 1:1 to 20:1, or 1:1 to 15:1, or 1:1 to 10:1, or 1:1 to 5:1 or 1:1 to 3:1, or about 1:1.
1.22 Any foregoing composition, wherein the composition comprises from 0.1 to 2% stannous fluoride, stannous chloride, or stannous pyrophosphate, by weight of the composition, e.g., 0.1 to 1.5%, or about 1%, or about 1%, about 0.1%, or about 0.2, or 0.2 to 0.75%.
1.23 Any foregoing composition, wherein the composition comprises from 0.1 to 5% of the nitric acid or water-soluble nitrate salt (e.g., potassium nitrate), by weight of the composition, e.g., 0.1 to 2%, or 0.1 to 1%, or 0.1 to 0.5%, or 0.2 to 0.4%, or 0.25 to 0.75%, about 0.3%, or about 0.5%.
1.24 Any foregoing composition, wherein the composition comprises from 0.1 to 5% of the polyphosphate source, wherein the polyphosphate source comprises three or more phosphate moieties (e.g., sodium hexametaphosphate, sodium trimetaphosphate, sodium tetrametaphosphate or phytic acid), by weight of the composition, e.g., 0.8 to 5%, or 0.8 to 4%, or 0.8 to 3%, or 0.8 to 2%, or 0.8 to 1.0%, or 1.0 to 1.5%, or about 0.8%, or about 1.2%.
1.25 Any foregoing composition, wherein the composition further comprises from 0.1 to 5% of an alkali metal polyphosphate salt (e.g., tetrasodium pyrophosphate or sodium tripolyphosphate), by weight of the composition, e.g., 0.8 to 5%, or 0.8 to 4%, or 0.8 to 3%, or 0.8 to 2%, or 0.8 to 1.0%, or 1.0 to 1.5%, or about 0.8%, or about 1.2%.
1.26 Any foregoing composition, wherein the composition comprises at least 10% water by weight of the composition, e.g., at least 20%, at least 30%, or at least 40%, or at least 50%, or at least 60% or at least 65%, up to 95% water, by weight of the composition, or about 20%, or about 30%, or about 40%, or about 60% or about 80%.
1.27 Any foregoing composition wherein the composition comprises 70% to 95% water, by weight of the composition, e.g., from 75% to 95%, or from 75% to 90%, or from 75% to 85%, or from 75% to 80%; or wherein the composition comprises from 10% to 50% water, by weight of the composition, e.g., 10% to 40%, or 10% to 30%, or about 20%.
1.28 Any foregoing composition, wherein the composition comprises one or more humectants (e.g., glycerin, sorbitol, propylene glycol, or a mixture thereof) in a net amount of 5% to 70% by weight of the composition, e.g., from 5% to 25% by weight of the composition, or from 10% to 25%, or from 15% to 25%, or about 20%, or from 30 to 70%, or from 35 to 60%, or from 40 to 60%, or from 60 to 70%, by weight of the composition.
1.29 Any foregoing composition, wherein the composition is a single phase, i.e., it does not form two phases on standing.
1.30 Any foregoing composition, wherein the composition is dual phase, i.e., it forms two phases on standing.
1.31 Composition 1.30, wherein the composition forms an emulsion immediately upon mixing, and separates into two phases upon standing within 10 minutes (e.g., within 5 minutes, or within 3 minutes, or within 1 minute).
1.32 Any foregoing composition, wherein the composition is a clear (e.g., not opaque or turbid) solution (e.g., not a suspension) or a clear (e.g., translucent, not opaque) semisolid or gel.
1.33 Any foregoing composition, wherein the composition is physically and chemically stable, for example, wherein no color change or precipitation occurs on storage at ambient conditions for 3 months or more (e.g., 6 months or more, or 1 year or more).
1.34 Composition 1.33, wherein the stannous ion concentration is substantially stable for at least three months on storage, e.g., the concentration of stannous ion is at least 80% of the original concentration, or at least 85%, or at least 90%.
1.35 Any foregoing composition, wherein the composition has a pH of between 5 and 9, or a pH between 6 and 8, or a pH between 6.5 and 7.5, or a pH between 6.9 and 7.1, or a pH of about 7.
1.36 Any foregoing composition, wherein the composition comprises less than 10% of any hydrophobic liquid or mixture of hydrophobic liquids (e.g., alkyl fatty acid esters (e.g., isopropyl myristate), vegetable oils, mineral oils, or combinations thereof), by weight of the composition, for example, less than 5% by weight or less than 3% by weight or less than 1% by weight, of such hydrophobic liquids.
1.37 Any foregoing composition, wherein the composition is free or substantially free of any hydrophobic liquid or mixture of hydrophobic liquids (e.g., less than 0.1% by weight of the composition).
1.38 Any of Compositions 1.0-1.35, wherein the composition comprises at least 10% of any hydrophobic liquid or mixture of hydrophobic liquids (e.g., alkyl fatty acid esters (e.g., isopropyl myristate), vegetable oils, mineral oils, or combinations thereof), by weight of the composition, for example, 10-90% by weight, or 20-80% by weight, or 30-70% by weight, or 30-50% by weight, or 10-50% by weight, or 10-30% by weight, of such hydrophobic liquids.
1.39 Any foregoing composition, further comprising a nonionic surfactant, e.g., a hydrophilic nonionic surfactant.
1.40 Composition 1.39, wherein the nonionic surfactant is a copolymer of ethylene oxide and propylene oxide, for example, a block copolymer (e.g., a triblock copolymer).
1.41 Composition 1.39, wherein the nonionic surfactant is a poloxamer, e.g., a triblock copolymer having a hydrophobic polypropylene glycol block flanked by hydrophilic polyethylene glycol blocks.
1.42 Composition 1.41, wherein the poloxamer has a polyethylene glycol block length of about 75 to 125 units (e.g., about 100-101), and a polypropylene block length of about 25 to 75 units (e.g., about 55-56), for example, poloxamer 407 or Pluronic F127.
1.43 Any foregoing composition, comprising a nonionic surfactant in an amount of 0.01 to 5.0%, by weight of the composition, e.g., 0.1 to 1.0%, 0.2 to 0.7%, 0.3 to 0.5%, about 0.4%.
1.44 Any foregoing composition, further comprising an anionic surfactant, e.g., selected from sodium laurel ether sulfate (SIRS), sodium lauryl sulfate, and ammonium lauryl sulfate.
1.45 Any foregoing composition wherein the composition further comprises one or more of a thickener (e.g., xanthan gum or carboxymethyl cellulose, such as sodium salt), a buffer, a sweetener, a flavorant, a pigment, a dye, an anti-caries agent, an anti-bacterial agent, a whitening agent, a desensitizing agent, a preservative, or a mixture thereof.
1.46 Any foregoing composition wherein composition further comprises an additional fluoride ion source.
1.47 Composition 1.46, wherein the additional fluoride ion source is selected from sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride) ammonium fluoride, titanium fluoride, hexafluorosulfate, or a mixture thereof.
1.48 Any foregoing composition wherein the composition comprises a whitening agent.
1.49 Any foregoing composition wherein the composition comprises a whitening agent, wherein the whitening agent is hydrogen peroxide.
1.50 Any foregoing composition wherein the composition further comprises a desensitizing agent selected from potassium chloride, strontium chloride, mixture thereof.
1.51 Any foregoing composition wherein the composition is a mouthwash.
1.52 Any foregoing composition wherein the composition is a dentifrice (e.g., a toothpaste or a tooth gel).
1.53 Any foregoing composition, wherein the composition is free of abrasives (e.g., the composition is free of silicas).
1.54 Any foregoing composition, wherein the composition comprises abrasive (e.g. silicas) in an amount of 1-30% by weight of the composition, e.g., 10-30%, or 20-25%, or 15-20%.
1.55 Any of the foregoing compositions, wherein the composition is effective upon application to the oral cavity, e.g., by rinsing, optionally in conjunction with brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing and/or malodor producing bacteria, (viii) treat, relieve or reduce dry mouth, (ix) clean the teeth and oral cavity, (x) whiten the teeth, (xi) reduce tartar build-up, (xii) reduce or prevent oral malodor, and/or (xiii) promote systemic health, including cardiovascular health, e.g., by reducing potential for systemic infection via the oral tissues.
1.56 Any foregoing composition, wherein the composition has enhanced stannous ion stability (e.g., compared to a composition comprising stannous fluoride or stannous chloride without both a nitrate ion source and a polyphosphate).
1.57 Any foregoing composition, wherein the composition is packaged in a container comprising a single storage compartment, which compartment comprises the composition, and a closure (e.g., a screw-top closure) which seals the compartment.
1.58 Any foregoing composition further comprising one or more of a zwitterionic surfactant (e.g., betaine), and a nonionic polymer (e.g., a polyethylene glycol, such as PEG-600).
1.59 Any foregoing composition, wherein the composition has less than 20% by weight of any one polymeric thickener (e.g., xanthan gum, carrageenan gum, carboxymethyl cellulose, such as sodium CMC), such as less than 15% by weight, or less than 10% by weight, or less than 5% by weight, or less than 1% by weight, or 0.05-1%, or 0.05-0.5%, or 0.25 to 0.75%, by weight, or about 0.5% by weight.
1.60 Any foregoing composition, wherein the composition has less than 40% by weight of any silica (e.g., thickening silica), such as 10-40%, or 10-30%, or 10-20% or 0-20%, or 0-10%, or about 15% by weight.
1.61 Any foregoing composition, wherein the composition is substantially transparent, e.g., having a % transmittance of visible light of 10-90% for a sample thickness of 20-25 mm (e.g., 15-50%), or >30-90% for a sample thickness of 15-20 mm, or 50-90% for a sample thickness of 10-15 mm, or 70-100% for a sample thickness of 5-10 mm.
1.62 Any foregoing composition, in the form of a gel having cylindrical cross section (e.g., diameter of 5-15 mm or 8-10 mm).
1.63 Any foregoing composition, in the form of a gel having a flat ribbon cross-section (e.g. with a thickness of 2-15 mm 5-10 mm).
1.64 Any of the foregoing compositions, wherein the compositions is a single-component oral care composition comprising:
   (i) stannous chloride, or stannous fluoride, or stannous pyrophosphate;
   (ii) potassium nitrate;
   (iii) sodium hexametaphosphate, or sodium trimetaphosphate, or sodium tetrametaphosphate or phytic acid; and
   (iv) more than 10% water, by weight of the composition.
1.65 The composition of 1.64, further comprising a water-soluble alkali metal polyphosphate (e.g., a sodium or potassium polyphosphate) (e.g, a water-soluble alkali metal polyphosphate selected from sodium pyrophosphate, potassium pyrophosphate, sodium tripolyphosphate and potassium tripolyphosphate.)
1.66 Any of the foregoing compositions further comprising 0.01 to 0.09% by weight of charcoal (e.g., activated charcoal); wherein the composition is formulated as a dentifrice (e.g., toothpaste or tooth gel).
1.67 The composition of 1.66, wherein the composition comprises 0.05 to 0.085% by weight of charcoal.
1.68 The composition of 1.66 or 1.67, wherein the composition comprises 0.05 to 0.08% or 0.06 to 0.08% by weight of charcoal.
1.69 The composition of any of 1.66-1.68, wherein the composition comprises 0.07 to 0.08% by weight of charcoal.
1.70 The composition of any of 1.66-1.69, wherein the composition comprises about 0.075% by weight of charcoal.
1.71 The composition of any of 1.66-1.70, wherein the charcoal is activated charcoal.
1.72 The composition of any of 1.66-1.71, wherein the composition has a light transmittance of at least 0.001% measured on a 10 mm-thick vertical sample, e.g., at least 0.01%, or at least 0.1%, or at least 0.2%, or 0.05% to 1%, or 0.1% to 1%, or 0.2% to 0.5%, or about 0.25%.
1.73 The composition of any of the foregoing compositions, wherein the phosphate source with three or more phosphate moieties or units comprises sodium hexametaphosphate, or sodium trimetaphosphate, or sodium tetrametaphosphate, or phytic acid, or combinations thereof.
1.74 The composition of any of the foregoing compositions, wherein sodium hexametaphosphate is the only phosphate source with three or more phosphate moieties or units.
1.75 The composition of any of the foregoing compositions, wherein sodium trimetaphosphate is the only phosphate source with three or more phosphate moieties or units.
1.76 The composition of any of the foregoing compositions, wherein sodium tetrametaphosphate is the only phosphate source with three or more phosphate moieties or units.
1.77 The composition of any of the foregoing compositions, wherein or phytic acid is the only phosphate source with three or more phosphate moieties or units.
1.78 The composition of any of the foregoing compositions, further comprising a basic amino acid (e.g., arginine).
1.79 The composition of any of the foregoing compositions, wherein the phosphate source with three or more phosphate moieties or units is in a range selected from: 3 to 50 units, 3 to 18 units, 4 to 50 units, 4 to 18 units, 6 to 50 units, or 6 to 18 units.
1.80 Any of the preceding compositions, wherein stannous fluoride is the only source of stannous ion in the composition.
1.81 Any of Composition 1.0-1.79, wherein stannous chloride is the only source of stannous ion in the composition.
1.82 Any of Composition 1.0-1.79, wherein stannous pyrophosphate is the only source of stannous ion in the composition.
1.83 Any of Composition 1.0-1.79, wherein stannous pyrophosphate and stannous fluoride are the only sources of stannous ion in the composition.
1.84 Any of Composition 1.0-1.79, wherein stannous chloride and stannous fluoride are the only sources of stannous ion in the composition.

In a second aspect, the present disclosure further provides a method (Method 1) of stabilizing stannous ion in an aqueous oral care composition comprising the steps of (1) providing an aqueous vehicle, (2) adding to the aqueous vehicle a stannous ion source, (3) adding to the aqueous vehicle a nitrate ion source, and (4) adding to the aqueous vehicle a polyphosphate ion source, wherein the polyphosphate ion source comprises three or more phosphate moieties or units ((e.g., from 3 to 50 units) (e.g., from 3 to 18 units) ((e.g., from 4 to 50 units) (e.g., from 4 to 18 units) (e.g., from 6 to 50 units) (e.g., from 6 to 18 units)), and wherein the final composition is a single-component high-water composition (e.g., at least 10% water).

For example, the disclosure provides embodiments of Method 1 as follows:
1.1 Method 1, wherein the stannous ion source is a water-soluble stannous salt.
1.2 Method 1 or 1.1, wherein the stannous salt is stannous fluoride, stannous chloride, or stannous pyrophosphate.
1.3 Any preceding method, wherein the nitrate ion source is nitric acid or a water-soluble nitrate salt.
1.4 Method 1.3, wherein the water-soluble nitrate salt is selected from an alkali or alkaline earth metal nitrate, or zinc nitrate, silver nitrate, or ammonium nitrate.
1.5 Method 1.3, wherein the water-soluble nitrate salt is an alkali metal nitrate salt or an alkaline earth metal nitrate salt.
1.6 Method 1.5, wherein the nitrate salt is selected from lithium nitrate, sodium nitrate, potassium nitrate, magnesium nitrate, and calcium nitrate.
1.7 Method 1.6, wherein the nitrate salt is potassium nitrate.
1.8 Any preceding method, wherein the polyphosphate source comprising three or more phosphate moieties is in a linear or ring arrangement, or pendants of a polymer.
1.9 Any preceding method, wherein the polyphosphate source comprising three or more phosphate moieties or units is a polyphosphate source where phosphates are pendent groups of a ring (e.g., phytic acid).
1.10 Any preceding method, wherein the polyphosphate source comprising three or more phosphate moieties is a phosphate source wherein the phosphates are pendent groups of a polymer (e.g., ethylene glycol methacrylate phosphate, vinylphosphonic acid, bis2-(methacryloyloxy)ethyl phosphate, 11-phosphonoundecyl acrylate, 2-methacryloyloxyethylphosphorcholine).
1.11 Any preceding method, wherein the wherein the polyphosphate source comprising three or more phosphate moieties is selected from: sodium trimetaphosphate, sodium tetrametaphosphate, sodium hexametaphosphate and combinations thereof.
1.12 Any preceding method, wherein the polyphosphate source comprising three or more phosphate moieties is selected from sodium hexametaphosphate, sodium trimetaphosphate, phytic acid and combinations thereof.
1.13 Any preceding method, wherein the method further comprises adding a water-soluble alkali metal polyphosphate.
1.14 Method 1.13, wherein the water-soluble alkali metal polyphosphate is selected from a pyrophosphate, tripolyphosphate, tetraphosphate or hexametaphosphate.
1.15 Method 1.14, wherein the water-soluble alkali metal polyphosphate is a sodium or potassium polyphosphate.
1.16 Method 1.15, wherein the water-soluble alkali metal polyphosphate is selected from sodium pyrophosphate, potassium pyrophosphate, sodium tripolyphosphate and potassium tripolyphosphate.
1.17 Method 1.16, wherein the sodium pyrophosphate salt is selected from sodium acid pyrophosphate (i.e., disodium pyrophosphate) and tetrasodium pyrophosphate.
1.18 Any preceding method, wherein the stannous salt is stannous fluoride or stannous chloride or stannous pyrophosphate, the nitrate salt is potassium nitrate, and the polyphosphate source is selected from: sodium trimetaphosphate, sodium tetrametaphosphate, sodium hexametaphosphate, phytic acid and combinations thereof.
1.19 Any preceding method, wherein the composition is formulated to have a molar ratio of polyphosphate source (e.g., sodium trimetaphosphate, sodium tetrametaphosphate, sodium hexametaphosphate, phytic acid) to stannous source (e.g., stannous fluoride, stannous chloride or stannous pyrophosphate) of at least 1:1, e.g., 1:1 to 5:1, or 1:1 to 4:1, or 1:1 to 3:1, or 1:1 to 2:1, or 1.5:1 to 5:1, or 2:1 to 5:1, or 2:1 to 4:1, or 2:1 to 3:1, or about 1:1.
1.20 Any preceding method, wherein the composition is formulated to have a molar ratio of nitric acid or nitrate source (e.g., potassium nitrate) to stannous source (e.g., stannous fluoride, stannous chloride or stannous pyrophosphate) of at least 0.01:1, (e.g., 0.01:1 to 20:1, 0.01:1 to 0.1:1, or 0.03:1, or 0.3:1 or 0.5:1 to 20:1, or 1:1 to 20:1, or 1:1 to 15:1, or 1:1 to 10:1, or 1:1 to 5:1 or 1:1 to 3:1, or about 1:1).
1.21 Any preceding method, wherein the composition is formulated to comprise from 0.1 to 2% stannous ion source (e.g., stannous fluoride, stannous chloride or stannous pyrophosphate), by weight of the composition, e.g., 0.1 to 1%, or 0.25 to 0.75%, or about 0.45%.
1.22 Any preceding method, wherein the composition is formulated to comprise from 0.1 to 5% of nitric acid or nitrate ion source (e.g., potassium nitrate), by weight of the composition, e.g., 0.1 to 2%, or 0.1 to 1%, or 0.1 to 0.5%, or 0.2 to 0.4%, or 0.25 to 0.75%, about 0.3%, or about 0.5%.
1.23 Any preceding method, wherein the composition is formulated to comprise from 0.1 to 5% of polyphosphate ion source, wherein the polyphosphate ion source comprises three or more phosphate moieties (e.g., sodium trimetaphosphate, sodium tetrametaphosphate, sodium hexametaphosphate, phytic acid), by weight of the composition, e.g., 0.8 to 5%, or 0.8 to 4%, or 0.8 to 3%, or 0.8 to 2%, or 0.8 to 1.0%, or 1.0 to 1.5%, or about 0.8%, or about 1.2%.
1.24 Any preceding method, wherein the aqueous vehicle comprises water and optionally one or more humectants (e.g., glycerin, sorbitol, propylene glycol, or a mixture thereof).
1.25 Any preceding method, wherein the composition is formulated to comprise from 10% to 95% water, by weight of the composition, e.g., from 20 to 95%, or from 30 to 95%, or from 40 to 95%, or from 50 to 95%, or from 60 to 95% or from 65 to 95%, by weight of the composition, or about 20%, or about 40%, or about 60% or about 80%.
1.26 Any preceding method, wherein the composition is formulated to comprise 70% to 95% water, by weight of the composition, e.g., from 75% to 95%, or from 75% to 90%, or from 75% to 85%, or from 75% to 80%; or wherein the composition is formulated to comprise from 10% to 50% water, by weight of the composition, e.g., 10% to 40%, or 10% to 30%, or about 20%.
1.27 Any preceding method, wherein the composition is formulated to comprise one or more humectants (e.g., glycerin, sorbitol, propylene glycol, or a mixture thereof) in a net amount of 5 to 75% by weight of the composition, e.g., from 5% to 25% by weight of the composition, or from 10% to 25%, or from 15% to 25%, or about 20%, or from 30 to 70%, or from 35 to 60%, or from 40 to 60%, or from 60 to 70%, by weight of the composition.
1.28 Any preceding method, wherein the composition is formulated as a single phase, i.e., it does not form two phases on standing.
1.29 Any preceding method, wherein the composition is formulated as a clear (e.g., not opaque or turbid) solution (e.g., not a suspension) or a clear (e.g., translucent, not opaque) semisolid or gel.
1.30 Any preceding method, wherein the composition is physically and chemically stable, for example, wherein no color change or precipitation occurs on storage at ambient conditions for 3 months or more (e.g., 6 months or more, or 1 year or more).
1.31 Method 1.27, wherein the stannous ion concentration is substantially stable for at least three months on storage, e.g., the concentration of stannous ion is at least 80% of the original concentration, or at least 85%, or at least 90%.
1.32 Any preceding method, wherein the composition has a pH of between 5 and 9, or a pH between 6 and 8, or a pH between 6.5 and 7.5, or a pH between 6.9 and 7.1, or a pH of about 7.
1.33 Any preceding method, wherein the composition is formulated to comprise less than 10% of any hydrophobic liquid or mixture of hydrophobic liquids (e.g., alkyl fatty acid esters (e.g., isopropyl myristate), vegetable oils, mineral oils, or combinations thereof), by weight of the composition, for example, less than 5% by weight or less than 3% by weight or less than 1% by weight, of such hydrophobic liquids.
1.34 Any preceding method, wherein the composition is formulated to be free or substantially free of any hydrophobic liquid or mixture of hydrophobic liquids (e.g., less than 0.1% by weight of the composition), i.e., the method does not comprise any step of adding any hydrophobic liquid to the aqueous vehicle.
1.35 Any preceding method, wherein the composition is formulated to comprise a nonionic surfactant, e.g., a hydrophilic nonionic surfactant, i.e., the method further comprises the step (5) of adding a nonionic surfactant to the aqueous vehicle.
1.36 Method 1.35, wherein the nonionic surfactant is a copolymer of ethylene oxide and propylene oxide, for example, a block copolymer (e.g., a triblock copolymer).
1.37 Method 1.35, wherein the nonionic surfactant is a poloxamer, e.g., a triblock copolymer having a hydrophobic polypropylene glycol block flanked by hydrophilic polyethylene glycol blocks.
1.38 Method 1.37, wherein the poloxamer has a polyethylene glycol block length of about 75 to 125 units (e.g., about 100-101), and a polypropylene block length of about 25 to 75 units (e.g., about 55-56), for example, poloxamer 407 or Pluronic F127.
1.39 Any of methods 1.35-1.38, wherein composition is formulated to comprise the nonionic surfactant in an amount of 0.01 to 5.0%, by weight of the composition, e.g., 0.1 to 1.0%, 0.2 to 0.7%, 0.3 to 0.5%, about 0.4% 1.40 Any preceding method, wherein the composition is a mouthwash.
1.41 Any preceding method, wherein the composition is a dentifrice (e.g., a toothpaste or a tooth gel).
1.42 Any preceding method, wherein the composition formulated to comprise abrasive (e.g. silicas) in an amount of 1-30% by weight of the composition, e.g., 10-30%, or 20-25%.
1.43 Any preceding method, wherein the composition is formulated to be free of abrasives (e.g., the composition is formulated to be free of silicas).
1.44 Any preceding method, wherein step (1) occurs first and steps (2)-(5) occur in any order.
1.45 Any preceding method, further comprising a final step (6) of packaging the composition in a container comprising a single storage compartment, which compartment comprises the composition, and a closure (e.g., a screw-top closure) which seals the compartment.
1.46 Any preceding method, wherein the method results in a composition according to Composition 1 et seq., (e.g., any of Composition 1.0-1.78).

In a third aspect, the present disclosure provides an oral care package comprising a composition according to Composition 1.0 et seq or Method 1 et seq wherein the package comprises a container comprising a single storage compartment, which compartment contains the composition, and a closure (e.g., a screw-top closure) which seals the compartment. In some embodiments, wherein the composition is a toothpaste or gel, the package comprises a closure which dispenses a ribbon of toothpaste or gel having a circular cross-section, oval cross-section, or flat-ribbon cross-section. In some embodiments, such ribbon is dispensed having a diameter or thickness of 5-25 mm, e.g., 5-10 mm, or 10-15 mm, or 15-20 mm, or 20-25 mm.

In a fourth aspect, the present disclosure provides a method of treatment or prevention of gingivitis, plaque, dental caries, and/or dental hypersensitivity, the method comprising the application to the oral cavity of a person in need thereof, of a composition according to the invention (e.g., Composition 1.0 et seq.), e.g., by brushing, for example, one or more times per day.

In a fifth aspect, the present disclosure provides a method (Method 2.0) where Method 2.0 is a method of treating or reducing malodor, in a subject in need thereof, and wherein the method comprises administering a composition of any of Composition 1.0 et seq to the oral cavity of the subject.

Alternatively, the present disclosure provides Composition 1.0, et seq., for use in the treatment or prevention of gingivitis, plaque, dental caries, malodor and/or dental hypersensitivity.

The methods of the fourth or fifth aspects comprise applying any of the compositions as described herein to the teeth, e.g., by brushing, gargling or rinsing, or otherwise administering the compositions to the oral cavity of a subject in need thereof. The compositions can be administered regularly, such as, for example, one or more times per day (e.g., twice per day). In various embodiments, administering the compositions of the present disclosure to teeth may provide one or more of the following specific benefits: (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing and/or malodor producing bacteria, (viii) treat, relieve or reduce dry mouth, (ix) clean the teeth and oral cavity, (x) whiten the teeth, (xi) reduce tartar build-up, (xii) reduce or prevent oral malodor, and/or (xiii) promote systemic health, including cardiovascular health, e.g., by reducing potential for systemic infection via the oral tissues.

In a sixth aspect, any of Composition 1.0 et seq can contain charcoal. In one aspect, it is expected that when cylindrical nurdles of a dentifrice (e.g., toothpaste or gel) of any of Composition 1.0 et seq, are measured in a 8-9 mm diameter, and have from 0.05 to 0.09% charcoal, those nurdles remain black and translucent. It will be appreciated that nurdles of different cross-section (e.g., flat ribbons, rectangular, oval) or nurdles of different thickness (e.g., less than 8 mm maximum thickness) will achieve black color and translucency at higher concentrations of charcoal (e.g., up to 0.15% charcoal). Analogously, it will be appreciated that nurdles of different cross-section or different thickness (e.g., more than 8 mm diameter) may maintain translucency and black color at lower charcoal concentrations (e.g., down to 0.01%).

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Oral care compositions include, for example, dentifrice and mouthwash. In some embodiments, the disclosure provides mouthwash formulations.

As used herein, “single component” means an oral care composition comprising at most a single compositional component at any time. Thus, this is in distinction to a “dual-component” compositions, which is manufactured as two separate compositions, maintained separately until final point of use. For example, a dual component toothpaste is typically packaged in a tube containing two parallel compartments exiting via a common nozzle such that when the user extrudes the toothpaste from the package the two components mix immediately prior to application to the oral cavity. Likewise, a dual component mouthwash is typically packaged in a bottle comprising two compartments such that a measured amount of the liquid from each compartment is dispensed and mixed when the user. Dual component compositions are often used to maintain in separate components and compartments ingredients which are mutually incompatible, such that if kept in the same component they would adversely react or interfere with each other.

In contrast, a dual-phase composition, such as a mouthwash, is a single-component composition comprising two immiscible liquids which settle into two phases on standing. Such a composition has no need for separated compartments for storage because the natural tendency of the two phases to separate helps ensure that the ingredients in one phase are not maintained in intimate contact with the ingredients of the other phase. Nevertheless, when vigorously mixed, the two phases become intimately combined (such as, to form an emulsion), which may or may not separate back into the two phases on standing.

### Fluoride Ion Source

In one aspect, the oral care compositions may further include one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., each of which are incorporated herein by reference. Representative fluoride ion sources used with the present invention (e.g., Composition 1.0 et seq.) include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof. Where the formulation comprises calcium salts, the fluoride salts are preferably salts wherein the fluoride is covalently bound to another atom, e.g., as in sodium monofluorophosphate, rather than merely ionically bound, e.g., as in sodium fluoride.

### Surfactants

In one aspect, the compositions of the disclosure, e.g., any of Composition 1.0 et seq., can contain anionic surfactants, for example, water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomo-glyceride sulfate; higher alkyl sulfates, such as sodium lauryl sulfate; higher alkyl-ether sulfates, e.g., of formula CH₃(CH₂)ₘCH₂(OCH₂CH₂)ₙOSO₃X, wherein m is 6-16, e.g., 10, n is 1-6, e.g., 2, 3 or 4, and X is Na or, for example sodium laureth-2 sulfate (CH₃(CH₂)₁₀CH₂(OCH₂CH₂)₂OSO₃Na); higher alkyl aryl sulfonates such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. By “higher alkyl” is meant, e.g., C₆₋₃₀ alkyl. In particular embodiments, the anionic surfactant (where present) is selected from sodium lauryl sulfate and sodium ether lauryl sulfate. When present, the anionic surfactant is present in an amount which is effective, e.g., >0.001% by weight of the formulation, but not at a concentration which would be irritating to the oral tissue, e.g., 1%, and optimal concentrations depend on the particular formulation and the particular surfactant. In one embodiment, the anionic surfactant is present at from 0.03% to 5% by weight, e.g., about 1.75% by wt.

In another embodiment, cationic surfactants useful in the present disclosure can be broadly defined as derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof. Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants of compositions of the disclosure, e.g., any of Composition 1.0, et seq., that can be used in the compositions of the invention can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials. In a particular embodiment, the composition of the invention comprises a nonionic surfactant selected from polaxamers (e.g., polaxamer 407), polysorbates (e.g., polysorbate 20), polyoxyl hydrogenated castor oils (e.g., polyoxyl 40 hydrogenated castor oil), and mixtures thereof.

Illustrative amphoteric surfactants of compositions of the disclosure, e.g., any of Composition 1.0, et seq., that can be used in the compositions of the invention include betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures of such materials.

The surfactant or mixtures of compatible surfactants can be present in the compositions of the present invention in 0.1% to 5%, in another embodiment 0.3% to 3% and in another embodiment 0.5% to 2% by weight of the total composition.

### Flavoring Agents

In one aspect, the compositions of the disclosure, e.g., any of Composition 1.0 et seq., may also include a flavoring agent. Flavoring agents which are used in the practice of the present invention include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint.

The flavoring agent is incorporated in the oral composition at a concentration of 0.01 to 1% by weight.

### pH Adjusting Agents

In some embodiments, the compositions of the present disclosure, e.g., any of Composition 1.0 et seq, contain a buffering agent. Examples of buffering agents include anhydrous carbonates such as sodium carbonate, sesquicarbonates, bicarbonates such as sodium bicarbonate, silicates, bisulfates, phosphates (e.g., monopotassium phosphate, monosodium phosphate, disodium phosphate, dipotassium phosphate, tribasic sodium phosphate, sodium tripolyphosphate, pentapotassium tripolyphosphate, phosphoric acid), citrates (e.g. citric acid, trisodium citrate dehydrate), pyrophosphates (sodium and potassium salts, e.g., tetrapotassium pyrophosphate) and combinations thereof. The amount of buffering agent is sufficient to provide a pH of about 5 to about 9, preferable about 6 to about 8, and more preferable about 7, when the composition is dissolved in water, a mouth rinse base, or a toothpaste base. Typical amounts of buffering agent are about 5% to about 35%, in one embodiment about 10% to about 30%, in another embodiment about 15% to about 25%, by weight of the total composition.

### Chelating and Anti-Calculus Agents

In one aspect, the compositions of the disclosure, e.g., any of Composition 1.0 et seq., may include one or more chelating agents able to complex calcium found in the cell walls of the bacteria. Binding of this calcium weakens the bacterial cell wall and augments bacterial lysis.

Another group of agents suitable for use as chelating or anti-calculus agents in the present invention are the soluble pyrophosphates. The pyrophosphate salts used in the present compositions can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The salts are useful in both their hydrated and unhydrated forms. An effective amount of pyrophosphate salt useful in the present composition is generally enough to provide at least 0.1 wt. % pyrophosphate ions, e.g., 0.1 to 3 wt. %, e.g., 0.1 to 2 wt. %, e.g., 0.1 to 1 wt. %, e.g., 0.2 to 0.5 wt %. The pyrophosphates also contribute to preservation of the compositions by lowering water activity.

Suitable anticalculus agents for the disclosure (e.g., any of Composition 1.0 et seq) include without limitation phosphates and polyphosphates (for example pyrophosphates), polyaminopropanesulfonic acid (AMPS), hexametaphosphate salts, zinc citrate trihydrate, polypeptides, polyolefin sulfonates, polyolefin phosphates, diphosphonates. In particular embodiments, the invention includes alkali phosphate salts, i.e., salts of alkali metal hydroxides or alkaline earth hydroxides, for example, sodium, potassium or calcium salts. “Phosphate” as used herein encompasses orally acceptable mono- and polyphosphates, for example, P₁₋₆ phosphates, for example monomeric phosphates such as monobasic, dibasic or tribasic phosphate; dimeric phosphates such as pyrophosphates; and multimeric phosphates, e.g., sodium hexametaphosphate. In particular examples, the selected phosphate is selected from alkali dibasic phosphate and alkali pyrophosphate salts, e.g., selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these. In a particular embodiment, for example the compositions comprise a mixture of tetrasodium pyrophosphate (Na₄P₂O₇), calcium pyrophosphate (Ca₂P₂O₇), and sodium phosphate dibasic (Na₂HPO₄), e.g., in amounts of ca. 3-4% of the sodium phosphate dibasic and ca. 0.2-1% of each of the pyrophosphates. In another embodiment, the compositions comprise a mixture of tetrasodium pyrophosphate (TSPP) and sodium tripolyphosphate (STPP)(Na₅P₃O₁₀), e.g., in proportions of TSPP at about 1-2% and STPP at about 7% to about 10%. Such phosphates are provided in an amount effective to reduce erosion of the enamel, to aid in cleaning the teeth, and/or to reduce tartar buildup on the teeth, for example in an amount of 2-20%, e.g., ca. 5-15%, by weight of the composition.

### Polymers

The oral care compositions of the disclosure, e.g., any of Composition 1.0 et seq., also optionally include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139(M.W. 500,000), AN 1 19 (M.W. 250,000) and S-97 Pharmaceutical Grade (M.W. 70,000), of GAF Chemicals Corporation.

Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

The N-vinyl-2-pyrrolidione is also commonly known as polyvinylpyrrolidone or “PVP”. PVP refers to a polymer containing vinylpyrrolidone (also referred to as N-vinylpyrrnlidone and N-vinyl-2-pyrrolidinone) as a monomeric unit. The monomeric unit consists of a polar imide group, four non-polar methylene groups and a non-polar methane group. The polymers include soluble and insoluble homopolymeric PVPs. Copolymers containing PVP include vinylpyrrolidone/vinyl acetate (also known as Copolyvidone, Copolyvidonum or VP-VAc) and vinyl pyrrolidone/dimethylamino-ethylmethacrylate. Soluble PVP polymers among those useful herein are known in the art, including Povidone, Polyvidone, Polyvidonum, poly(N-vinyl-2-pyrrolidinone), poly (N-vinylbutyrolactam), poly(l-vinyl-2-pyrrolidone) and poly [1-(2-oxo-1 pyrrolidinyl)ethylene]. These PVP polymers are not substantially cross-linked. In some embodiments the polymer comprises an insoluble cross-linked homopolymer. Such polymers include crosslinked PVP (often referred to as cPVP, polyvinylpolypyrrolidone, or cross-povidone).

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

In preparing oral care compositions, it is sometimes necessary to add some thickening material to provide a desirable consistency or to stabilize or enhance the performance of the formulation. In certain embodiments, the thickening agents are carboxyvinyl polymers, carrageenan, xanthan, hydroxyethyl cellulose and water soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 5.0% by weight of the total composition are used.

In some embodiments, microcrystalline cellulose (MCC) can be used (e.g., carboxymethyl cellulose with sodium carboxymethyl cellulose). An example of a source of MCC is Avicel® (FMC Corporation), which contains MCC in combination with sodium carboxymethyl cellulose (NaCMC). Both Avicel®. RC-591 (MCC containing 8.3 to 13.8 weight % NaCMC) and Avicel®. CL-611 (MCC containing 11.3 to 18.8 weight % NaCMC) may be used in certain aspects. In certain embodiments, the ratio of microcrystalline cellulose to cellulose ether thickening agent is from 1:1 to 1:3 by weight; or from 1:1.5 to 1:2.75 by weight. In any of the above embodiments comprising sodium carboxymethylcellulose, microcrystalline cellulose may be used in combination with NaCMC. In certain such embodiments, the MCC/sodium carboxymethylcellulose may be present in an amount of from 0.5 to 1.5 weight % based on the total weight of the composition.

### Abrasives

Natural calcium carbonate is found in rocks such as chalk, limestone, marble and travertine. It is also the principle component of egg shells and the shells of mollusks. The natural calcium carbonate abrasive of the invention is typically a finely ground limestone which may optionally be refined or partially refined to remove impurities. For use in the present invention, the material has an average particle size of less than 10 microns, e.g., 3-7 microns, e.g. about 5.5 microns. For example a small particle silica may have an average particle size (D50) of 2.5-4.5 microns. Because natural calcium carbonate may contain a high proportion of relatively large particles of not carefully controlled, which may unacceptably increase the abrasivity, preferably no more than 0.01%, preferably no more than 0.004% by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ.

Precipitated calcium carbonate is generally made by calcining limestone, to make calcium oxide (lime), which can then be converted back to calcium carbonate by reaction with carbon dioxide in water. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 325 mesh. The particles may for example have a D50 of 3-6 microns, for example 3.8=4.9, e.g., about 4.3; a D50 of 1-4 microns, e.g. 2.2-2.6 microns, e.g., about 2.4 microns, and a D10 of 1-2 microns, e.g., 1.2-1.4, e.g. about 1.3 microns. The particles have relatively high water absorption, e.g., at least 25 g/100 g, e.g. 30-70 g/100 g. Examples of commercially available products suitable for use in the present invention include, for example, Carbolag® 15 Plus from Lagos Industria Quimica.

In certain embodiments the invention may comprise additional calcium-containing abrasives, for example calcium phosphate abrasive, e.g., tricalcium phosphate (Ca₃(PO₄)₂), hydroxyapatite (Ca₁₀(PO₄)₆(OH)₂), or dicalcium phosphate dihydrate (CaHPO₄·2H₂O, also sometimes referred to herein as DiCal) or calcium pyrophosphate, and/or silica abrasives, sodium metaphosphate, potassium metaphosphate, aluminum silicate, calcined alumina, bentonite or other siliceous materials, or combinations thereof. Any silica suitable for oral care compositions may be used, such as precipitated silicas or silica gels. For example synthetic amorphous silica. Silica may also be available as a thickening agent, e.g., particle silica. For example, the silica can also be small particle silica (e.g., Sorbosil AC43 from PQ Corporation, Warrington, United Kingdom). However the additional abrasives are preferably not present in a type or amount so as to increase the RDA of the dentifrice to levels which could damage sensitive teeth, e.g., greater than 130.

### Amino Acids

In one aspect, any of the compositions of the disclosure, e.g., any of Compositions 1.0 et seq., can include a basic or neutral amino acid. The basic amino acids which can be used in the compositions and methods of the invention include not only naturally occurring basic amino acids, such as arginine, lysine, and histidine, but also any basic amino acids having a carboxyl group and an amino group in the molecule, which are water-soluble and provide an aqueous solution with a pH of 7 or greater.

For example, basic amino acids include, but are not limited to, arginine, lysine, serine, citrullene, ornithine, creatine, histidine, diaminobutanoic acid, diaminoproprionic acid, salts thereof or combinations thereof. In a particular embodiment, the basic amino acids are selected from arginine, citrullene, and ornithine.

In certain embodiments, the basic amino acid is arginine, for example, L-arginine, or a salt thereof.

In another aspect, the compositions of the disclosure (e.g., any of Compositions 1.0 et seq) can include a neutral amino acid, which can include, but are not limited to, one or more neutral amino acids selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and combinations thereof.

The compositions of the disclosure (e.g., any of Composition 1.0 et seq) are intended for topical use in the mouth and so salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

### Water

Water is present in the oral compositions of the disclosure, e.g., any of Composition 1.0, et seq. Water, employed in the preparation of commercial oral compositions should be deionized and free of organic impurities. Water commonly makes up the balance of the compositions and includes 5% to 45%, e.g., 10% to 20%, e.g., 25-35%, by weight of the oral compositions. This amount of water includes the free water which is added plus that amount which is introduced with other materials such as with sorbitol or silica or any components of the invention. The Karl Fischer method is a one measure of calculating free water.

### Humectants

Within certain embodiments of the oral compositions of the disclosure, e.g., any of Composition 1.0 et seq., it is also desirable to incorporate a humectant to reduce evaporation and also contribute towards preservation by lowering water activity. Certain humectants can also impart desirable sweetness or flavor to the compositions. The humectant, on a pure humectant basis, generally includes 15% to 70% in one embodiment or 30% to 65% in another embodiment by weight of the composition.

Suitable humectants include edible polyhydric alcohols such as glycerin, sorbitol, xylitol, propylene glycol as well as other polyols and mixtures of these humectants. Mixtures of glycerin and sorbitol may be used in certain embodiments as the humectant component of the compositions herein.

The present invention in its method aspect involves applying to the oral cavity a safe and effective amount of the compositions described herein.

The compositions and methods according to the disclosure (e.g., Composition 1.0 et seq) can be incorporated into oral compositions for the care of the mouth and teeth such as dentifrices, toothpastes, transparent pastes, gels, mouth rinses, sprays and chewing gum.

Other ingredients which may optionally be included in compositions according to the present disclosure include hyaluronic acid, green tea, ginger, sea salt, coconut oil, turmeric, white turmeric (white curcumin), grape seed oil, ginseng, monk fruit, vitamin E, basil, chamomile, pomegranate, aloe vera, and charcoal. Any of such ingredients may be present in an amount from 0.01% to 2% by weight of the composition, e.g., 0.01 to 1%, or 0.01 to 0.5%, or 0.01 to 0.1%.

### EXAMPLES

Unless otherwise noted, all figures for stannous ion concentration refer to soluble stannous, not total stannous (total stannous being soluble and insoluble stannous combined).

### Example 1—Stannous Stability and Cyclic Polyphosphates with at Least Three Phosphate Moieties

An accelerate aging study is conducted. Sn (II) stability after two weeks @ 60° C. accelerated aging of solutions containing 0.454 wt. % SnF₂, 0.3 wt. % KNO₃, and a molar equivalent of the below cyclic polyphosphates (Test Samples: A, B, and C) is listed in Table 1 below. Identical solutions containing linear tetrasodium pyrophosphate (TSPP) listed for comparison.

TABLE 1

| | Wt. % for 1 molar | % remaining |
|---|---|---|
| | equivalent | Sn (II) |
| Test Sample A: Sodium | 1.8 | 38% |
| Hexametaphosphate | | |
| Test Sample B: Sodium | 0.9 | 91% |
| Trimetaphosphate | | |
| Test Sample C: Phytic Acid | 1.9 | 40% |
| Linear Tetrasodium | 0.77 | 87% |
| Pyrophosphate (Control) | | |

### Example 2—Transparent Dentifrice Formulations

It is also expected that compositions made according to the present disclosure, especially toothpaste or gel compositions, will be surprisingly translucent. Without being bound by theory, it is believed that the presence of un-solubilized stannous ion in a high-water dentifrice may contribute significantly to opacity. It therefore believed that the solubilization of stannous ion according to the present disclosure (by interaction with nitrate and polyphosphate ions) removes this impediment to clarity and transparency. A properly formulated dentifrice composition according to the present disclosure is expected to achieve substantial improvements in clarity and transparency compared to prior dentifrice compositions.

1. An oral care composition comprising:
stannous ion source selected from stannous fluoride, stannous chloride, stannous pyrophosphate and a combination of two or more thereof;
nitric acid, a water-soluble nitrate salt, or a combination of two or more thereof;
polyphosphate source, wherein the polyphosphate source comprises three or more phosphate moieties or units; and
more than about 10 wt. % of water, by weight of the composition.

2. The oral care composition according to claim 1, wherein the water-soluble nitrate salt is selected from an alkali metal nitrate, alkaline earth metal nitrate, zinc nitrate, silver nitrate, ammonium nitrate, and a combination of two or more thereof.

3. The oral care composition according to claim 1, wherein the water-soluble nitrate salt is selected from lithium nitrate, sodium nitrate, potassium nitrate, magnesium nitrate, calcium nitrate, and a combination of two or more thereof.

4. The oral care composition of according to claim 1, wherein the water-soluble nitrate salt comprises potassium nitrate.

5. The oral care composition according claim 1, wherein the polyphosphate source comprising three or more phosphate moieties has a linear arrangement, a ring arrangement, and/or arranged as pendants of a polymer.

6. The oral care composition according to claim 1, wherein the polyphosphate source comprising three or more phosphate moieties is a polyphosphate source where phosphates are pendent groups of a ring.

7. The oral care composition according to claim 1, wherein the polyphosphate source comprising three or more phosphate moieties is selected from sodium trimetaphosphate, sodium tetrametaphosphate, sodium hexametaphosphate, phytic acid, and combinations thereof.

8. The oral care composition according to claim 1 further comprising a water-soluble alkali metal polyphosphate selected from a pyrophosphate, tripolyphosphate, tetraphosphate, hexametaphosphate, and a combination thereof.

9. The oral care composition according to claim 8, wherein the water-soluble alkali metal polyphosphate is selected from sodium pyrophosphate, potassium pyrophosphate, sodium tripolyphosphate, potassium tripolyphosphate, and a combination of two or more thereof.

10. The oral care composition according to claim 1, wherein the composition comprises a molar ratio of the polyphosphate source comprising three or more phosphate moieties to the stannous source of about 1:1 to about 1:5.

11. The oral care composition according to claim 1, wherein the composition comprises a molar ratio of the total of nitric acid and/or water-soluble nitrate salt to the stannous ion source is about 0.3:1 to about 20:1.

12. The oral care composition according to claim 1, wherein the oral care composition comprises from about 0.1 to about 2 wt. % of the stannous ion source; from about 0.1 to about 5 wt. % of the nitric acid, the water-soluble nitrate salt, or the combination of two or more thereof; from about 0.1 to about 5 wt. % of the polyphosphate source comprising three or more phosphate moieties, wherein all weight percentages are based on the total weight of the oral care composition.

13. The oral care composition according to claim 1, wherein the composition comprises from 10 to 95 wt. % of water, by weight of the composition.

14. (canceled)

15. (canceled)

16. The oral care composition according to claim 1, wherein the oral care composition further comprises one or more humectants in an amount from 5 to 25 wt. %, by weight of the composition.

17. The oral care composition according to claim 1, wherein the oral care composition is a single-phase composition.

18. The oral care composition according to claim 1, wherein the oral care composition is in the form of a single component, dual phase composition.

19.-22. (canceled)

23. The oral care composition according to claim 1, wherein the compositions is a single-component oral care composition comprising:
(i) stannous chloride, or stannous fluoride, or stannous pyrophosphate;
(ii) potassium nitrate;
(iii) sodium hexametaphosphate, or sodium trimetaphosphate, or sodium tetrametaphosphate or phytic acid; and
(iv) more than 10% water, by weight of the composition.

24. An oral care package comprising an oral care composition according to claim 1, wherein the package comprises a container comprising a single storage compartment, which compartment contains the oral care composition, and a closure which seals the compartment.

25. A method of treatment or prevention of gingivitis, plaque, dental caries, dental hypersensitivity and/or malodor, the method comprising the application to the oral cavity of a person in need thereof, of an oral care composition according to claim 1.

26. A method of stabilizing stannous ion in an aqueous oral care composition comprising the steps of (1) providing an aqueous vehicle, (2) adding to the aqueous vehicle a stannous ion source, (3) adding to the aqueous vehicle a nitrate ion source, and (4) adding to the aqueous vehicle a polyphosphate ion source wherein the polyphosphate ion source comprises three or more phosphate moieties or units, wherein the final composition is a single-component high-water composition.
